(19) United States
(12) Patent Application Publication
(10) Pub. No.: US 2017/0307485 A1
(43) Pub. Date: Oct. 26, 2017

(54) REFRACTIVE INDEX MATCHING COMPOSITION FOR BIOLOGICAL TISSUE

(71) Applicant: LOGOS BIOSYSTEMS, INC, Anyang (KR)

(72) Inventor: Neon Cheol Jung, Anyang (KR)

(21) Appl. No.: 15/492,171

(22) Filed: Apr. 20, 2017

(30) Foreign Application Priority Data
Apr. 20, 2016 (KR) 10-2016-0048247

(51) Int. Cl. G01N 1/30; G01N 1/36; G01N 1/40; G01N 33/53; G01N 1/28

(52) U.S. Cl. CPC G01N 1/30; G01N 33/53; G01N 1/36; G01N 1/286; G01N 1/40; G01N 2001/2873; G01N 2001/4038

(57) ABSTRACT

The present invention is about biological tissue refractive index (RI) matching composition and the method for clearing biological tissue. Specifically, the RI matching composition of the present invention shows remarkable RI matching effects when clearing biological tissue, and has excellent fluorescent signal preservation, and can be used for long-term storage of biological tissue since the biological tissue remains clear during in low temperature storage.

### CROSS-REFERENCE TO RELATED APPLICATIONS

This patent application claims priority to KR Patent Application No. 10-2016-0048247, filed Apr. 20, 2016, the content of which is incorporated herein by reference in its entirety.

### TECHNICAL FIELD

The present invention is about a refractive index matching composition for biological tissue. Specifically, this application relates to a refractive index matching composition comprising iopromide as an effective ingredient and the method for optically clearing biological tissue using thereof.

### BACKGROUND ART

To observe a thick biological tissue with an optical microscope, the tissue should be sliced into thin sections (≦20 mm) to permit light transmission therethrough for obtaining the images thereof, and then the images of the respective sections should be three-dimensionally reconstructed by computer program. Such manipulating process requires labor and long time to prepare thousands to tens of thousands of thin sections or slices and to reconstruct the images, according to the size of the biological tissue. Further, it is not easy to combine exactly the large amount of information of the thin sections. Therefore, it is considered that the most preferable way for 3-D imaging of biological tissue is to acquire images of the intact tissue rather than preparing thin slices, and thus various biological tissue clearing methods have been developed.

The biological tissue clearing technology aims to provide a method for three-dimensionally observing optically non-transparent thick biological tissue with an optical microscope, instead of cutting them into thin slices or sections. For this purpose, a method for removing certain components in biological tissue, a method for chemically homogenizing the different refractive indexes (RI) of the biological components, such as, proteins, lipids, and carbohydrates, etc., and the combination of the said two methods have been suggested. For example, fluorescence labeled antibodies or various kinds of fluorescent probes are embedded into the optically transparent tissue, and thereafter three-dimensional images of the tissue are procured using a confocal microscope.

A representative biological tissue clearing method, which removes certain biological components from a living organism, is the method of removing lipids with an organic solvent. Saplteholz published a method for removing lipids by dissolving them in benzylbenzoate in 1914, as the first biological tissue clearing technology (Spalteholz, W. (1914). Uber das Durchsichtigmachen von menschlichen and tierischen Praparaten (S. Hierzel)). Then, the improved technologies, for example, BABB (Dodt, H. U., Leischner, U., Schierloh, A., Ja hrling, N., Mauch, C. P., Deininger, K., Deussing, J. M., Eder, M., Zieglga nsberger, W., and Becker, K. (2007)) and 3DISCO (Ertu rk, A., Becker, K., Ja hrling, N., Mauch, C. P., Hojer, C. D., Egen, J. G., Hellal, F., Bradke, F., Sheng, M., and Dodt, H. U. (2012a). Three-dimensional imaging of solvent-cleared organs using 3DISCO. Nat. Protoc. 7, 1983-1995) emerged.

Scale et al. published a different biological tissue clearing technology using highly concentrated non-solvent compounds, such as, urea, glycerol, and sucrose etc., to minimize the possible protein denaturation which could be caused by organic solvents (Hama, H., Kurokawa, H., Kawano, H., Ando, R., Shimogori, T., Noda, H., Fukami, K., Sakaue-Sawano, A., and Miyawaki, A. (2011). Scale: a chemical approach for fluorescence imaging and reconstruction of transparent mouse brain. Nat. Neurosci. 14, 1481-1488), or CUBIC (Susaki, E. A., Tainaka, K., Perrin, D., Kishino, F., Tawara, T., Watanabe, T. M., Yokoyama, C., Onoe, H., Eguchi, M., Yamaguchi, S., et al. (2014). Whole-brain imaging with single-cell resolution using chemical cocktails and computational analysis. Cell 157, 726-739.). Further, the CLARITY technology was introduced. According to the CLARITY, certain components in biological tissue are replaced by exogenous material of hydrogel to maintain the intact structure of the tissue, and then electrophoresis is performed to remove lipids from the tissue (Chung, K., Wallace, J., Kim, S. Y., Kalyanasundaram, S., Andalman, A. S., Davidson, T. J., Mirzabekov, J. J., Zalocusky, K. A., Mattis, J., Denisin, A. K., et al. (2013). Structural and molecular interrogation of intact biological systems. Nature 497, 332-337.).

Further, it was known that appreciably optically transparent biological tissue could be prepared by simply storing the tissue sections in a solution comprising certain chemical compounds for a long time, if the tissue sections are not thick. The chemical compounds, which can be used for this method, include, for example, diatrizoic acid, fructose, thioglycerol, formamide, and PEG, etc. (Chiang, A. S., Lin, W. Y., Liu, H. P., Pszczolkowski, M. A., Fu, T. F., Chiu, S. L., and Holbrook, G. L. (2002). Insect NMDA receptors mediate juvenile hormone biosynthesis. Proc. Natl. Acad. Sci. USA 99, 37-42., Kuwajima, T., Sitko, A. A., Bhansali, P., Jurgens, C., Guido, W., and Mason, C. (2013). SeeDB: a simple and morphologypreserving optical clearing agent for neuronal circuit reconstruction. Nat. Neurosci. 16, 1154-1161.).

Though the biological tissue clearing methods mentioned above gave rise to fairly transparent tissues, refractive index (RI) homogenization process was still required, since it was not possible to exactly match the RI values of the tissue components only with such methods (Susaki, E. A., Tainaka, K., Perrin, D., Kishino, F., Tawara, T., Watanabe, T. M., Yokoyama, C., Onoe, H., Eguchi, M., Yamaguchi, S., et al. (2014). Whole-brain imaging with single-cell resolution using chemical cocktails and computational analysis. Cell 157, 726-739., Chung, K., Wallace, J., Kim, S. Y., Kalyanasundaram, S., Andalman, A. S., Davidson, T. J., Mirzabekov, J. J., Zalocusky, K. A., Mattis, J., Denisin, A. K., et al. (2013). Structural and molecular interrogation of intact biological systems. Nature 497, 332-337., Yang, B., Treweek, J. B., Kulkarni, R. P., Deverman, B. E., Chen, C. K., Lubeck, E., Shah, S., Cai, L., and Gradinaru, V. (2014). Single-cell phenotyping within transparent intact tissue through whole-body clearing. Cell 158, 945-958).

If the refractive indexes of the biological tissue components are not homogenized, the tissue-penetrating light is scattered, which causes such problems as image distortion, resolution decrease during imaging process due to the laser scattering, and imaging depth decrease resulting from laser transmission decrease. In this regard, various kinds of chemical compounds, which were used for biological tissue clearing, have been also used for the refractive index homogenization process. The reagent for the RI homogenization comprises, for example, FocusClear (trademark, Cell Explorer, Taiwan) of which major component is diatrizoic acid, and RIMS of which major component is Hhistodenz (trademark, Sigma-Aldrich) (Yang, B., Treweek, J. B., Kulkarni, R. P., Deverman, B. E., Chen, C. K., Lubeck, E., Shah, S., Cai, L., and Gradinaru, V. (2014). Single-cell phenotyping within transparent intact tissue through whole-body clearing. Cell 158, 945.958.). Such reagents provide large number of electrons to the tissue-penetrating light so that the whole tissue can be homogenized to have higher refractive indexes.

Regarding the RI matching compositions disclosed in the documents referenced above, the fluorescent material expressed by any interesting indicator in the biological tissue cannot be maintained more than one day when benzylbenzoate is used for the composition (BABB technology), and immunofluorescence staining is limited when dibenzylether is employed (3DISCO technology) (Ertu rk, A., Nat. Protoc. 7, 1983.1995.). Further, it was indicated that FocusClear and RIMS protocols have problems with high cost and the lack of long-term preservation of fluorescent signal, etc. (Yang, B., Treweek, J. B., Kulkarni, R. P., Deverman, B. E., Chen, C. K., Lubeck, E., Shah, S., Cai, L., and Gradinaru, V. (2014). Single-cell phenotyping within transparent intact tissue through whole-body clearing. Cell 158, 945-958.).

Further, the FocusClear technology using diatrizoic acid has the problem of using expensive raw materials, and the RIMS technology using histodenz has a problem in that crystallines are developed in low temperature, thereby preventing high resolution imaging.

### SUMMARY

In this regard, the technical problems to be solved of the present invention is to provide biological tissue refractive index matching composition which can be produced cost effectively by a simple preparing method, and is able to rapidly homogenize biological tissue refractive indexes, and can preserve fluorescent signals of the marked fluorescent probes, while not causing damage to the tissue. Specifically, this invention provides a refractive index matching composition comprising iopromide as an active ingredient.

Another purpose of the present invention is to provide a method for preparing biological tissue refractive index matching composition.

It is yet another purpose of the present invention to provide a composition for mounting biological tissue. The composition for mounting biological tissue is embedded into the tissue or its fragments (sections or slices), thereby preventing dehydration and preserving transparency thereof.

It is yet another purpose of the present invention to provide a method for biological tissue clearing.

In order to achieve the purposes above, this invention provides a composition comprising iopromide or its active derivatives as effective ingredients. The iopromide has the following chemical structure of Formula (1).

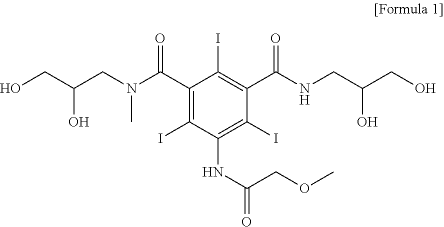 [SMILES: COCC(=O)Nc1c(I)c(C(=O)NCC(O)CO)c(I)c(C(=O)N(C)CC(O)CO)c1I]

In one embodiment, the biological tissue RI matching composition of the present invention may comprise 0.1 g to 5 g, preferably 0.15 g to 3 g of iopromide in 1 ml of the solvent. In the present invention, the composition can be prepared by dissolving iopromide in a solvent, and then stirring the mixture at between 10° C. and 50° C., preferably at between 20° C. and 40° C.

In another embodiment, the composition for mounting biological tissue further comprises antibodies, fluorescent probes, or nucleic acids, in addition to the biological tissue refractive index matching composition of the present invention.

In yet another embodiment, the method for clearing biological tissue comprises,

(i) embedding hydrogel into biological tissue in vitro separated from a living organism;
(ii) removing lipids from the biological tissue embedded with the hydrogel; and
(iii) submerging the delipidated biological tissue in a composition comprising iopromide.
The lipids can be removed from the tissue by any conventional lipid removing method, for example, washing or electrophoresis. When the lipids are removed by electrophoresis, the electrophoresis process can be performed with a commercially available or a self-manufactured device for hours, if necessary for the purpose. When the lipids are removed by washing, the biological tissue can be incubated in a detergent. The detergent can be selected from the group consisting of triton-X, saponin, NP-40 (Nonyl phenoxypolyethoxylethanol), tween20 and SDS (Sodium dodecyl sulfate), but not limited thereto.

The above biological tissue clearing method may further comprise extracting biological tissue and slicing it into sections. In addition, the method may further comprise immunolabeling the biological tissue before submerging it in the composition comprising iopromide.

The biological tissue RI matching composition of the present invention has excellent refractive index matching effects, and has remarkable fluorescent signal preservation ability comparing to the commercially available reagent of FocusClear (trademark). Further, there is almost no denaturation of the biological tissue treated with the composition of the present invention. Further, the present invention makes it possible to preserve biological tissue for a long time, by solving the problem of returning to non-transparent status during the storage in low temperature for the tissues treated with RIMS during preservation at low temperature.

### BRIEF DESCRIPTION OF DRAWINGS

FIG. 1 shows the result of RI matching of biological tissue by using the present RI homogenization composition, after imbedding acrylamide into the lung tissues of a mouse and then removing lipids therefrom using electrophoresis.

FIG. 2 shows the refractive indexes according to the concentration of iopromide in the RI homogenization composition of the present invention.

FIG. 3 is a photo representing the recovery times of the tissues swollen by biological tissue clearing.

FIG. 4 shows the relative size and light intensity of the swollen tissues according to times.

A: the comparison of tissue sizes before/after the tissue clearing; and

B: the comparison of light intensities before/after the tissue clearing

FIG. 5 shows the preservation, 2 weeks after the biological tissue clearing with the present RI homogenization composition.

FIG. 6 shows the transparency preservation during the storage in low temperature, after the biological tissue clearing by using the present composition.

### DETAILED DESCRIPTION

Hereinafter, the present invention is explained in more detail with the examples. However, the present invention is not subjected to any limitations in the following description.

In one aspect, the present invention is about a biological tissue RI homogenization composition comprising iopromide as an active ingredient.

The present biological tissue RI homogenization composition is used for a method for clearing tissue or its fragments (slices or sections). That is, the present invention relates to a RI matching composition which is used for RI homogenization of the components of biological tissue or its fragments.

The words, “refractive index homogenization”, “refractive index matching” and “RI matching” mean clearing tissue by homogenizing refractive indexes of the tissue components.

The “active derivative” of the present invention comprises, without limitation, chemical compounds which have refractive index homogenization effects comparable to those of iopromide.

The “biological tissue clearing” means to make a tissue transparent to three dimensionally observe it or its fragments with an optical microscope. The biological tissue clearing technology comprises, for example, CLARITY and PARS technologies. According to the CLARITY, the hydrogel (for example, acrylamide) is embedded into the tissue, followed by delipidation using electrophoresis, and then the refractive index is homogenized to make the tissue transparent. Further, according to the PARS, the hydrogel (for example, acrylamide) is embedded into the tissue and then SDS buffer solution is circulated thorough the tissue for a long time to have the tissue transparent.

The tissue specimen made transparent by the refractive index matching composition of the present invention can be detected with an optical microscope, a fluorescence microscope, a confocal microscope, a single-photon microscope or two-photon microscope, or a light-sheet microscope.

In one embodiment, iopromide is the following chemical compound of Formula 1 or its active derivatives:

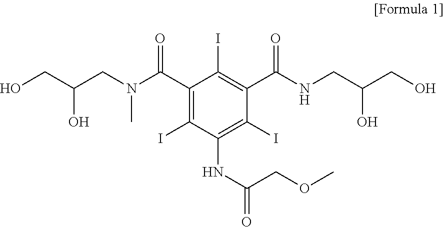 [SMILES: COCC(=O)Nc1c(I)c(C(=O)NCC(O)CO)c(I)c(C(=O)N(C)CC(O)CO)c1I]

1-N,3-N-bis(2,3-dihydroxypropyl)-2,4,6-triiodo-5-(2-methoxyacetamido)-1-N-methylbenzene-1,3-dicarboxamide

In one embodiment, the refractive index homogenization composition of the present invention comprises 0.15 g to 3 g of iopromide in 1 ml of a solvent which is DDW (double distilled water), PBS (phosphate buffered saline), saline solution (Normal saline) or ethanol, but not limited thereto.

In one aspect, the present invention is about a method for preparing a refractive index homogenization composition.

In one embodiment, the refractive homogenization composition for biological tissue can be prepared by dissolving iopromide in a solvent and then stirring the mixture at between 20° C. and 40° C.

In one aspect, the present invention relates to a tissue mounting composition which comprises the refractive index homogenization composition for biological tissue.

The words, “composition for mounting a tissue” mean a mounting solution to fill in biological tissue for imaging thereof, since the tissue or its fragments left as they are loose transparency due to dehydrogenation.

The composition for mounting a tissue can be prepared by additionally adding glycerin, gelatin, rubber syrup, balsam, or biolite to the composition. In addition, antibodies, fluorescent probes or nucleic acids can be further included for detecting specific materials.

In one aspect, the present invention relates to a method for clearing biological tissue, which comprises, i) embedding hydrogel into a tissue in vitro separated from a living organism or a tissue of an animal except human beings; ii) removing lipids from the tissue embedded with the hydrogel; iii) adding a biological tissue refractive index homogenization composition to the delipidated tissue.

In one embodiment, the removal of lipids can be performed by electrophoresis, and the electrophoresis can be carried out for hours or for days, if necessary, by the use of commercially available devices or self-manufactured devices.

In one embodiment, the delipidation can be performed by incubating the tissue in a detergent but not electrophoresis, and the detergent can be selected from the group consisting of triton-X, saponin, NP-40, tween20 and SDS (Sodium dodecyl sulfate). For example, 1 to 8% SDS buffer can be employed.

In another embodiment, the method for clearing biological tissue comprises, i) perfusing a tissue in vitro separated from a living organism or a tissue of an animal except human being by using hydrogel; ii) extracting the tissue perfused with the hydrogel; and iii) removing lipids from the extracted tissue, iv) submersing the tissue above iii) in the refractive index homogenization composition for biological tissue of the present invention.

In yet another embodiment, the extracted biological tissue is further processed to fragments.

In yet another embodiment, immunolabeling process is further performed before submerging tissue samples in the biological tissue refractive index matching composition of the present invention. Antibodies, fluorescent probes, nucleic acids or proteins can be used for the immunolabeling.

The present invention will be further explained based on the examples hereunder. The examples hereunder, however, are only for the purpose of illustrating the present invention, and the scope of the present invention is not limited to the examples hereunder.

### Example

### Preparation 1: Preparation of a Biological Tissue Refractive Index Matching Composition Comprising Iopromide

35 g of iopromide (Ultravist, Bayer Healthcare) was dissolved and mixed in 30 ml of DDW (double distilled water)

### Example 1: Biological Tissue Clearing by Using the Biological Tissue RI Matching Composition of the Preparation 1 Above

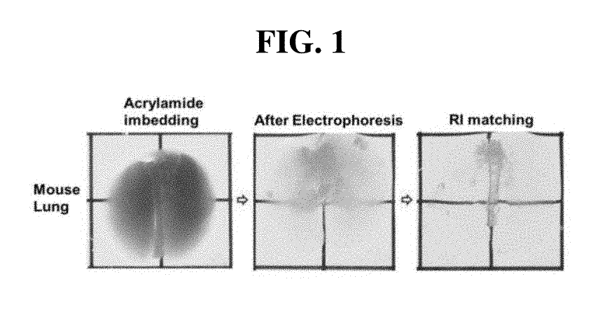

The lung of a mouse (ICR, female, 12 week old, Daehanbiolink Co., Ltd.) was extracted, and then the extracted lung was imbedded with hydrogel solution comprising arylamide for 24 hrs at 4° C., and thereafter the polymerization of hydrogel solution was allowed for 3 hrs at 37° C. Next, electrophoresis was carried out for the lung having the polymerized acrylamide by using the X-CLATRITY (Trademark, Logosbiosystems Inc., Korea). The specific operating conditions for the electrophoresis was as follows: 15 A, 60V, 4% SDS buffer solution, 30 rpm, for 12 hrs. After completing the electrophoresis, the lung tissues were treated with 5 ml of the RI matching solution prepared above, for 300 hrs, at room temperature to clear the tissues. Subsequently, the lung tissues of the mouse were detected with a stereoscopic microscope (Nikon, SMZ 745T, photos were taken by EP1 camera, Olympus). The results show that the composition of the present invention has excellent refractive index matching effects (see FIG. 1).

### Example 2: Assessment of Refractive Index According to the Concentration of Iopromide in the RI Matching Composition

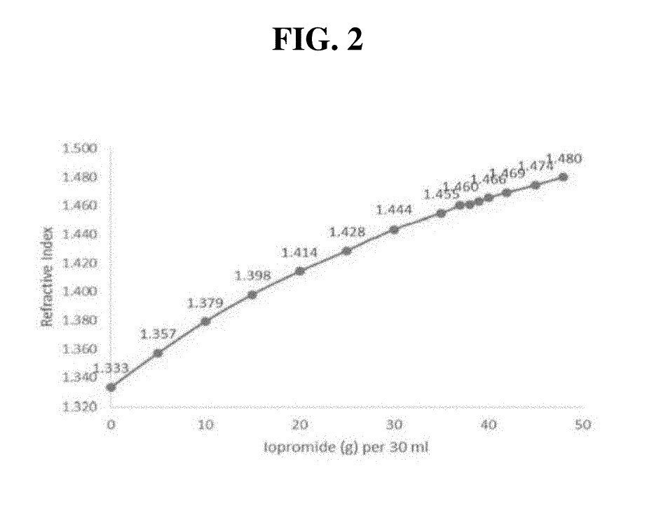

As shown in Table 1 below, 0 (control), 5 g, 10 g, 15 g, 20 g, 25 g, 30 g, 35 g, 37 g, 38 g, 39 g, 40 g, 42 g, 45 g or 48 g of iopromide was dissolved and mixed well in 30 ml of DDW at 25° C. or 37° C., to prepare refractive index matching compositions having different iopromide concentrations. The lung tissues were cleared with the RI matching compositions having different iopromide concentrations as described in the Example 1, and then the refractive indexes were assessed. The RIs were detected with a refractive index detector (ATAGO, RX-9000a). As a result, the RI increased in proportion to the concentration of iopromide in the RI matching composition (see Table 1 and FIG. 2).

TABLE 1

| iopromide | | |
|---|---|---|
| (g)/DDW 30 ml | Refractive Index (R.I) | dissolving condition (° C.) |
| 0 | 1.333 | 25° C. |
| 5 | 1.357 | 25° C. |
| 10 | 1.379 | 25° C. |
| 15 | 1.398 | 25° C. |
| 20 | 1.414 | 25° C. |
| 25 | 1.428 | 25° C. |
| 30 | 1.444 | 25° C. |
| 35 | 1.455 | 25~37° C. |
| 37 | 1.460 | 37° C. |
| 38 | 1.461 | 37° C. |
| 39 | 1.463 | 37° C. |
| 40 | 1.466 | 37° C. |
| 42 | 1.469 | 37° C. |
| 45 | 1.474 | 37° C. |
| 48 | 1.480 | 37° C. |

*DDW: double distilled water

### Example 3: Comparison of the Recovery Times of the Tissues Swollen after Tissue Clearing Treatments

As disclosed in the Example 1, the lung tissues of mouse was perfused with acrylamide, and then lipids were removed by electrophoresis. Next, the tissue clearing was carried out by submerging the tissues in the RI matching composition of the present invention, or the conventional RI matching composition of RIMS (Yang et al., 2014) or FocusClear (Chiang et al., 2002, Cell Explorer, Taiwan). Then, tissue recovery was assessed by determining the size of the swollen tissue at each time point of 0 hr (control), 0.5 hr, 1 hr, 2 hrs, and 3 hrs after the completion of the tissue clearing, and detecting the light transmission intensities.

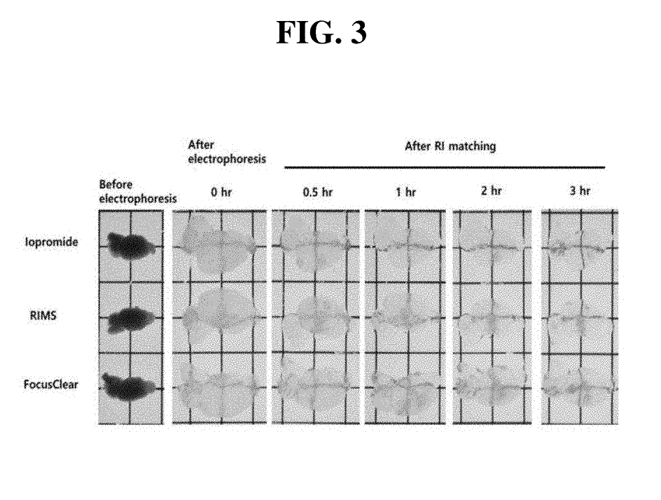

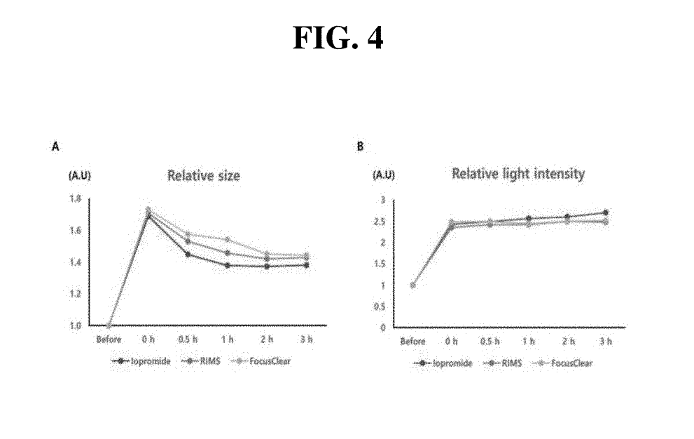

As a result, regarding the recovery effects of returning the size of the biological tissues swollen by the tissue clearing to the original size, the RI matching composition comprising iopromide of the present invention was remarkable in comparison to the composition of RIMS or FocusClear (FIG. 3 and FIG. 4A). Further, the tissues, which were cleared with the RI matching composition comprising iopromide of the present invention, showed the most excellent light transmission property (FIG. 4B).

Therefore, it was confirmed that the composition comprising iopromide of the present invention has significantly outstanding RI matching effects, in comparison to the conventional RI matching composition (FocusClear or RIMS).

### Example 4: Confirmation of Fluorescent Signal Preservation

Lipids were removed from the mouse brain tissue slices (1 mm thick) by using 4% SDS solution and electrophoresis device as disclosed in the above Example 1, and then the primary antibody (anti-collagen IV, Bio Rad Company) staining was performed at 37° C. for 24 hrs. Next, the primary antibodies were washed off with PBS, and the antibodies (Cy-3, Bio Rad Company) labeled with the second fluorescent materials were stained under the same conditions as for the primary antibodies. The washed sample was made to be transparent by matching the RI with the RI composition of the present invention, RIMS or FocusClear, and thereafter the tissue staining was assessed and imaged using a confocal microscope (ZEISS, LSM 710). Subsequently, after 2 weeks, the preservation of the fluorescent staining was confirmed under the same experimental conditions as above.

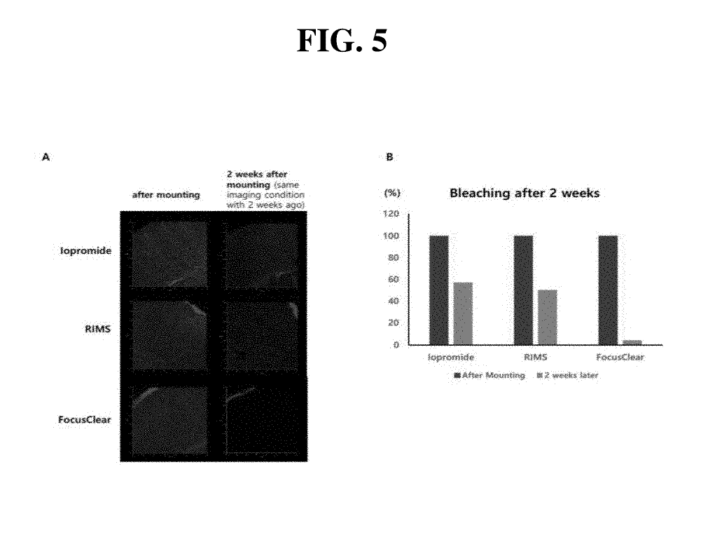

As a result, while the fluorescent signal of the tissue treated with the FocusClear for the tissue clearing was rapidly decreased, the tissues treated with the composition comprising iopromide minimized the fluorescence bleaching, thereby presenting excellent fluorescence preservation capacity (FIG. 5).

### Example 5: Assessment of the Maintenance of Transparency During in Low Temperature Storage

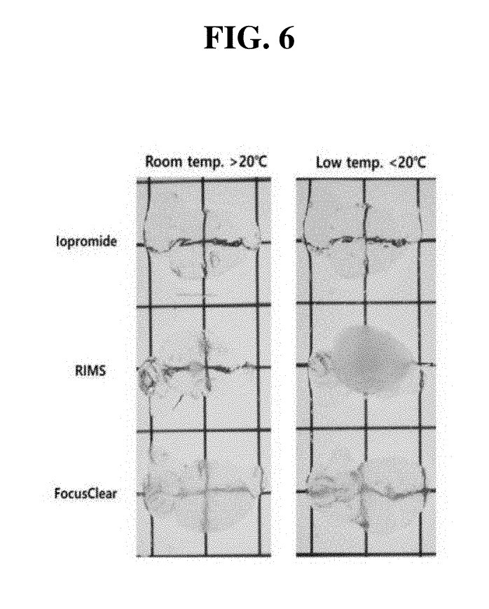

As disclosed in Example 1, the RI matching composition of the present invention, RIMS or FocusClear was used to make the lung tissues of a mouse optically transparent. After the tissue clearing treatment, the tissues were immersed in 5 ml of each RI matching composition and stored for 24 hrs, at 25° C. and 18° C., and then the tissue clearing was quantified with a stereoscopic microscope (Nikon, SMZ 745T, equipped with Olympus, EP1 camera). As a result, crystalline materials were developed in the biological tissue stored in the RIMS, which resulted in rapid decrease of the RI of the tissue. To the contrary, no crystalline material was detected in the biological tissue stored in the RI composition of the present invention (see FIG. 6).

1. A refractive index matching composition for biological tissue, which comprises iopromide or its active derivative as an active ingredient.

2. The refractive index matching composition of claim 1, wherein the iopromide has the following chemical structure of Formula 1:

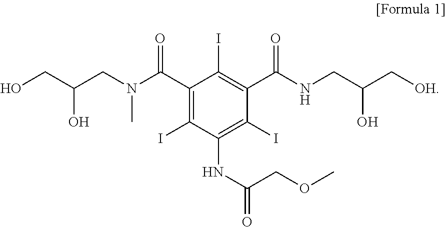 [SMILES: COCC(=O)Nc1c(I)c(C(=O)NCC(O)CO)c(I)c(C(=O)N(C)CC(O)CO)c1I]

3. The refractive index matching composition of claim 1, wherein the composition comprises 0.1 g to 5 g of iopromide in 1 ml of a solvent.

4. A method for preparing a refractive index matching composition for biological tissue, which comprises dissolving and stirring iopromide in a solvent.

5. The method for preparing a refractive index matching composition for biological tissue of claim 4, wherein the stirring is conducted at between 20° C. and 40° C.

6. A composition for mounting biological tissue, which comprises the refractive index matching composition of claim 1.

7. The composition for mounting biological tissue of claim 6, which further comprises antibodies, fluorescent probes, or nucleic acids.

8. A tissue clearing method which comprises,
i) embedding hydrogel into in vitro separated biological tissue or biological tissue of an animal except human beings;
ii) removing lipids from the tissue embedded with the hydrogel; and
iii) submerging the delipidated tissue into the composition of claim 1.

9. The tissue clearing method of claim 8, wherein lipids are removed by electrophoresis.

10. The tissue clearing method of claim 9, wherein the electrophoresis is carried out for hours to days.

11. The tissue clearing method of claim 8, wherein the lipids are removed by incubating the tissue in a detergent.

12. The tissue clearing method of claim 11, wherein the detergent is selected from the group consisting of triton-X, saponin, NP-40 (nonyl phenoxypolyethoxylethanol), tween20 and SDS (sodium dodecyl sulfate).

13. The tissue clearing method of claim 8, which further comprises slicing the tissue into sections, after extracting the biological tissue.

14. The tissue clearing method of claim 8, which further comprises immunolabeling the biological tissue, before submerging the tissue in the composition of claim 1.
